(19) United States
(12) Patent Application Publication
(10) Pub. No.: US 2005/0037031 A1
(43) Pub. Date: Feb. 17, 2005

(54) Methods for diet and weight control by altering the senses of smell and taste

(76) Inventor: Milton S. Jackson, Campbellsville, KY (US)

Correspondence Address: KIRTON AND MCCONKIE;1800 EAGLE GATE TOWER, 60 EAST SOUTH TEMPLE, P O BOX 45120, SALT LAKE CITY, UT, 84145-0120, US

(21) Appl. No.: 10/640,508

(22) Filed: Aug. 12, 2003

(51) Int. Cl. A61K 47/00; A61K 9/00

(52) U.S. Cl. 424/400

(57) ABSTRACT

This invention comprises a method for diet and weight control by altering the senses of smell and taste. The topically applied composition hinders the use of the olfactory nerves by either chemical or mechanical means. A further embodiment comprises a scent diffuser that blocks a food's smell. The loss of smell and taste caused by the composition and devices effectively decreases the appetite of an individual, enabling the individual to eat less and lose weight.

### 1. PRIORITY CLAIMED

This application claims priority to provisional patent application 60/403,066, filed Aug. 12, 2002.

### BACKGROUND OF THE INVENTION

1. Field of the Invention

The present invention relates to diet and weight control. More particularly, the present invention relates to methods for diet and weight control by altering the senses of smell and taste.

2. Background and Related Art

Obesity is a major health care issue. Sixty to eighty percent of American adults are considered overweight and 20-40% of these are considered morbidly obese. Many children and teenagers are also overweight. Obesity is one of the most important health issues in the world today because of its prevalence and close relation to other health issues such as hypertension, diabetes, strokes, heart diseases, pulmonary diseases, cancers, orthopedic problems (e.g. back, hip and knee pain) and many other serious and debilitating diseases. Obesity and related conditions cost Americans tens of billions of dollars annually and shorten peoples' life spans.

Obesity also impacts a person's quality of life. For instance, a person's general activity, exercise tolerance, and the ability to perform physical work are functions of weight. A person's self esteem is often related to one's physical appearance. As a result, people spend millions of dollars on clothing, accessories, and cosmetics, often in order to look better.

A person's weight is dictated by a simple physical relationship between his/her inputs (food) and outputs (metabolism, growth and exercise). Too much input or too little output can produce weight gain. Generally, if a person's input decreases, he/she loses weight.

Hunger and appetite are related to many things, including smells, tastes, habits, social issues, time issues and activity levels. Taste is experienced through inputs from both the tongue and nose. A person's tongue enables one to feel the texture of food and to tell whether food is sweet, salty, sour or bitter. A person's nose enables one to experience and differentiate between close to 10,000 different odors. The combination of input from both the tongue and nose enables the brain to have a combined sensory input that we refer to as taste. Since the tongue is limited to sensing only the texture, sweetness, saltiness, sourness and bitterness of food, however, a person's nose adds more quality to the taste experience than the tongue.

### SUMMARY OF THE INVENTION

The object of the present invention is to diet and control one's caloric intake by altering the sense of smell and thereby limiting or alerting the taste experience. Implementation of the present invention takes place in association with compositions and devices that alter the senses of smell and taste. The composition is topically applied either as a cream in the nose, or as a desiccated powder that is activated in the nasal cavity to shield the olfactory nerves from exposure to the smells in the nose. In a further implementation, the topically applied composition temporarily chemically hinders the effectiveness of the olfactory nerves. In another implementation, the composition comprises a scent diffuser that blocks a food's smell. In another implementation, a mechanical device blocks the effectiveness of the olfactory nerves.

The loss of smell caused by the composition and devices effectively decreases the appetite of an individual, encouraging the individual to eat less.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

The present invention relates to diet and weight control. More particularly, the present invention relates to methods for diet and weight control by altering the senses of smell to reduce appetite.

In the disclosure and in the claims the term “composition” shall refer to any substance or preparation that affects the senses of smell and taste. The present invention can be described by the following examples.

### EXAMPLE 1

An individual with a 3000-calorie per day diet topically applies a creme composition to the surfaces of the nasal cavity, thereby physically shielding the olfactory nerves from contact with the smells in the nose.

### EXAMPLE 2

An individual with a 3500-calorie per day diet topically applies a composition that chemically deadens the olfactory nerves thereby reducing their effectiveness.

### EXAMPLE 3

An individual with a 3300-calorie per day diet uses nose plugs to physically block the smell of food.

Thus, as discussed herein, the embodiments of the present invention embrace methods for diet and weight control by altering or prohibiting the sense of smell and its influence on the taste experience.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A method for diet and weight control comprising the step of:
applying to the interior of the nasal cavity a cream which physically blocks communication between the smells and the olfactory nerves in the nasal cavity.

2. A method for diet and weight control comprising the steps of:
moistening the interior of the nasal cavity with misted water;
inhaling through the nose a powdered desiccated adhesive;
eating at least one meal;
administered a decongestant; and
removing the adhesive from the nasal cavity.

3. A method for diet and weight control comprising the step of:
applying to the interior of the nasal cavity a cream which chemically prevents the olfactory nerves from functioning, thereby blocking communication between the smells and the olfactory nerves in the nasal cavity.

4. A method for diet and weight control comprising the step of:
blocking access to the nasal cavity by inserting nasal plugs when eating to physically block communication with the olfactory nerves in the nasal cavity.

5. A method for diet and weight control comprising the steps of:
applying to the interior of the nasal cavity a scent which masks the odors in the nasal cavity to reduce or alter communication between the odors and the olfactory nerves in the nasal cavity.
